(19) United States
(12) Patent Application Publication
(10) Pub. No.: US 2026/0263264 A1
(43) Pub. Date: Sep. 10, 2026

(54) Protective Heating Sheet for a Device for Transporting Human Beings

(71) Applicants: ILO Technology, Cremieu (FR); Advanced Healthcare Technology Limited, Royaume-Uni (GB)

(72) Inventors: Christophe Bietrix, Cremieu (FR); Mark Lait, Hertford (FR)

(21) Appl. No.: 19/139,747

(22) PCT Filed: Dec. 13, 2023
(86) PCT No.: PCT/EP2023/085711

§ 371 Date: Apr. 17, 2026

(30) Foreign Application Priority Data
Dec. 16, 2022 (FR) 2213574

(51) Int. Cl. A61F 7/00; A61F 7/02; A61G 11/00

(52) U.S. Cl. CPC A61F 7/0097; A61G 11/00; A61F 2007/0071; A61F 2007/0244

(57) ABSTRACT

The invention relates to a protective heating sheet intended to be removably fastened in a transport device intended to maintain the temperature of a human being, in particular a newborn, comprising a thin-film heating element with a web of electrically conductive means and at least one layer made of an electrically insulating material intended to be connected to a power supply, wherein A1 the electrically conductive means are made of a conductive material, the resistance of which continuously varies as a function of the temperature, at least one face of the heating element being at least partially covered with a covering layer made of a textile material. The invention also relates to a device for transporting human beings, in particular newborns, using such a sheet.

Abrégé: L'invention concerne une feuille protectrice chauffante destinée à être fixée de maniére amovible dans un dispositif de transport destiné à maintenir en température un être humain et notamment un nouveau-né, comprenant un élément chauffant en couche mince avec des moyens conducteurs électriques en nappe et au moins une couche réalisée en un matériau électriquement isolant, destiné à être relié à une alimentation électrique, dans lequel lesdits moyens conducteurs électriques sont réalisés en un matériau conducteur dont la résistance varie de façon continue en fonction de la température, au moins une face dudit élément chauffant étant recouverte au moins partiellement d'une couche de revêtement en un matériau textile, ainsi qu'un dispositif de transport d'êtres humains et notamment

The invention concerns the technical field of means for maintaining the temperature of human beings used in particular during their transfer from one place to another in a medical context.

These means can equally be used in incubators intended for newborns and that must not only prevent any bacterial contamination but also assure a constant temperature to prevent any stress on fragile organisms.

Numerous means have already been proposed for heating devices receiving human beings, whether they are intended for transporting them or not.

There may in particular be cited document EP3041450 which describes a transportable newborn incubator in which heating is provided by a flow of air passing through perforations provided in the bottom of the device.

The document EP1374005 also describes an incubator including heating devices and fans for circulating warm air inside the incubator. It describes means for regulation of the volume of air that circulates to limit noise and losses by evaporation.

It remains that these heating devices based on circulation of air have the disadvantages of being noisy, which contributes to the stress of the newborns, leading to losses by evaporation, requiring the presence of sensors that need regular maintenance, and consuming a large quantity of energy. These devices are also bulky and heavy and because of this compromise the safety of the newborn in the event of an accident.

The document WO2010/107724 describes an incubator in which a heating mattress is placed. The latter includes a heating element incorporated in a thick layer of foam or gel.

This incubator enables a newborn to be kept warm without circulation of air but necessitates sterilization of the mattress between two uses, which leads to numerous manipulations and necessitates large storage spaces.

The invention has for object alleviating these disadvantages by proposing a protective heating sheet intended in use to be removably fixed into a device for maintaining the temperature of a human being, which temperature can be maintained at a precise setpoint value that can be adapted as a function of use, while avoiding the presence of sensors in the sheet, which enables simplification of its design and eliminates any input of warm air from the outside.

This heating sheet enables the temperature of a human being to be maintained by supplying heat thereto as close as possible without losses. In practise this sheet is fixed to a padding provided in the maintained temperature device, in particular when it is a transport device.

This simplified design also means that the protection sheet can be for single use, because of its resulting low cost of manufacture. Now, the possibility of single use is often imperative in a medical context.

This protection function enables sterilization of the padding of the transport device between two uses to be avoided.

Thus the invention concerns a protective heating sheet intended in use to be removably fixed into a device for maintaining the temperature of a human being and in particular a newborn, including a thin layer heating element with flat electrically-conductive means and at least one layer made of an electrically-insulative material intended to be connected to an electrical power supply, wherein said electrically-conductive means are made of a conductive material the resistance of which varies continuously as a function of temperature, at least one face of said heating element being at least partly covered by a textile material covering layer.

Advantageous embodiments may further have recourse to one and/or the other of the following arrangements:
the heating element of said sheet is a heating film, the electrically-conductive means consisting in a metal conductive track positioned over at least a central and main part of the sheet;
the heating sheet is sterile;
the heating sheet includes at least one fixing tab in said temperature maintaining device linked to the main part of the sheet and projecting from said main part, said tab having a face provided with a layer of adhesive;
the heating sheet is for single use;
said at least one fixing tab is linked to said main part by a tear line;
the heating sheet includes a connecting zone to be connected to said electrical power supply, linked to said main part and projecting relative to said main part, a conductive track extending over this connecting zone and ending at two ends;
the connecting zone includes means for positioning an electrical connector.

The invention also concerns a kit comprising at least one protective heating sheet according to the invention and an electrical power supply control module intended to be connected between the heating sheet and an electrical power supply, said module including means for determining the resistance of the conductive means of the heating element, deducing therefrom the temperature Tt of the woven material at a time t, comparing it to a setpoint value Tc, and controlling the electrical power supply as a function of said comparison.

This kit may include an electrical connector between the heating sheet and the module, said connector being removable.

The invention also concerns a device for maintaining the temperature of human beings and in particular a device for transporting newborns, including a box delimiting an enclosure that forms a housing for a human being, the temperature of which is regulated by means of a heating sheet on which the human being can be placed, avoiding circulation of warm air.

This enables simplification of the environment and the maintenance of the device since all input of warm air from the outside is eliminated. The removal of the means generating warm air enables a considerable reduction of the weight of the device. This constitutes a considerable advantage in risk management, in particular when transporting a newborn.

Thus the invention concerns a device for maintaining the temperature of human beings and in particular of newborns, in particular a transport device, including two parts delimiting an enclosure, one of them forming a housing for a human being and including a heating sheet according to the invention disposed in said housing.

It finally concerns a device for transporting human beings and in particular newborns, including two parts delimiting an enclosure, one forming a housing for a human being and including a kit according to the invention, said heating sheet being disposed in said housing and said module being outside said enclosure.

This design enables dangerous and bulky objects to be placed outside the enclosure.

Such a device advantageously includes padding a or mattress to which the heating sheet can be removably fixed.

The invention finally concerns the use of a protective heating sheet according to the invention in a device, in particular a transport device, for maintaining the temperature of human beings, in particular newborns.

The invention will be better understood and other aims, advantages and features thereof will become more clearly apparent on reading the following description, which is given with reference to the appended drawings, in which:

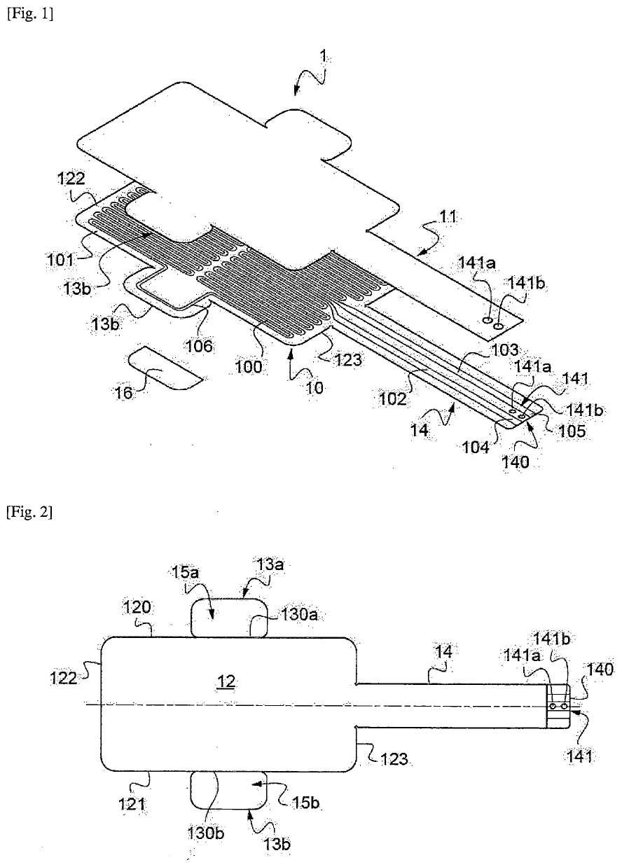

FIG. 1 is a schematic exploded perspective view of one embodiment of a heating sheet according to the invention.

FIG. 2 is a view from below of the sheet depicted in FIG. 1.

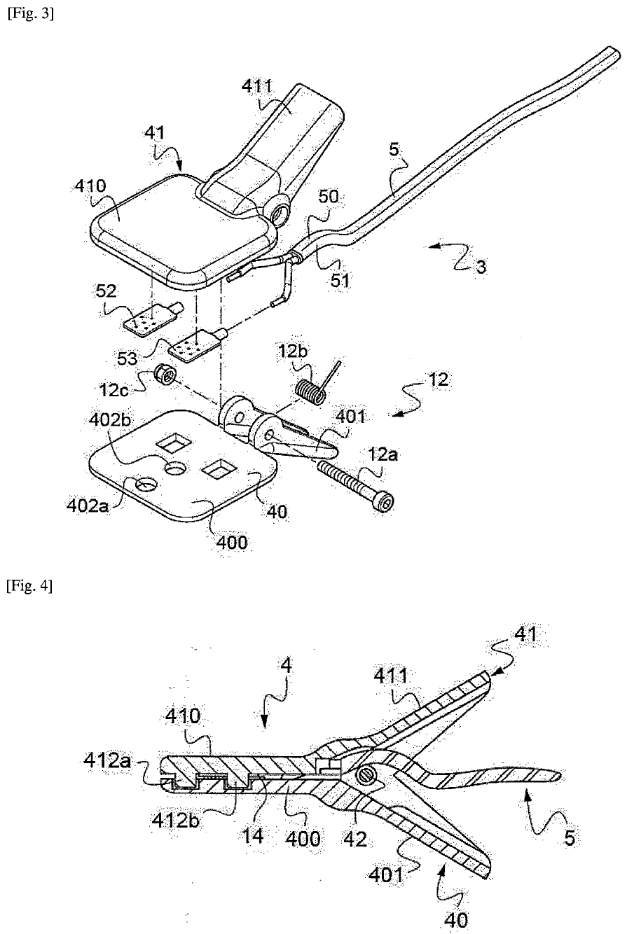

FIG. 3 is a schematic exploded perspective view of one embodiment of an electrical connector between the sheet depicted in FIGS. 1 and 2 and a regulation module.

FIG. 4 is a side view in perspective depicting the electrical connector when closed on the connecting zone of the sheet depicted in FIGS. 1 and 2.

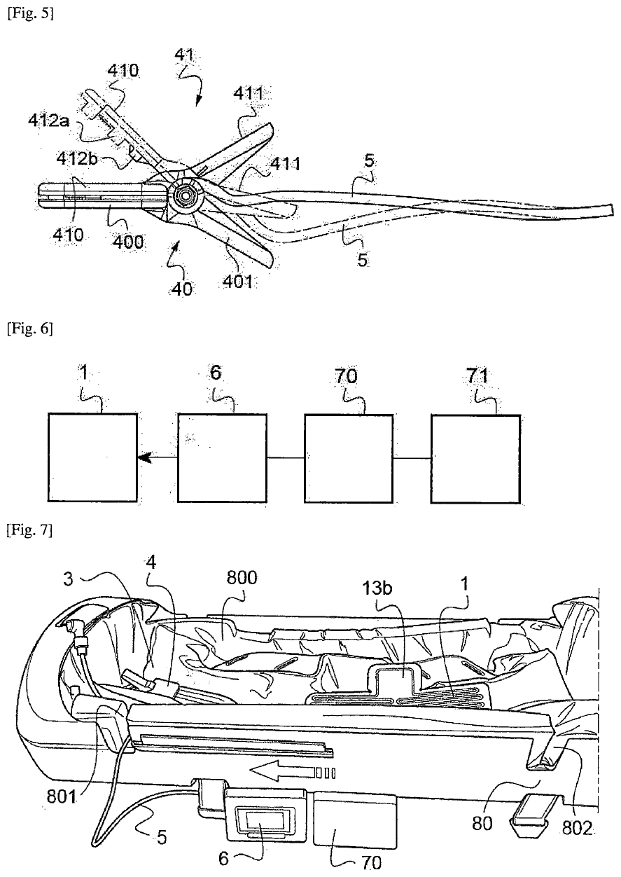

FIG. 5 is a side view in section depicting (in solid line) the electrical connector when closed and (in chain-dotted line) when open.

FIG. 6 is a general schematic showing the arrangement of a heating sheet and a control module in use.

FIG. 7 is a perspective view depicting a device for transporting newborns in which a heating sheet according to the invention has been placed.

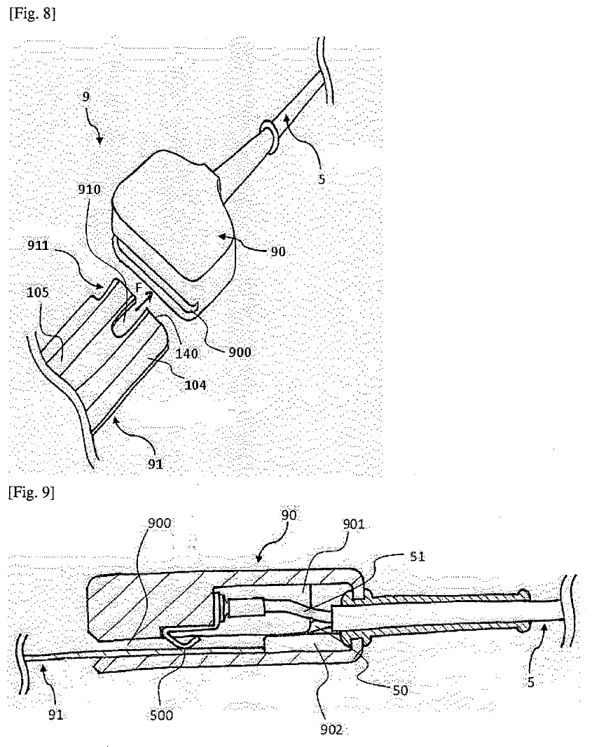

FIG. 8 is a perspective view of another example of an electrical connector between the sheet depicted in FIGS. 1 and 2 and a regulation module represented when open.

FIG. 9 is a side view in section depicting the electrical connector when closed.

Elements common to the various figures are designated by the same references.

The protective sheet 1 depicted in FIG. 1 includes a thin layer heating element 10 that is covered by a textile material layer 11. These two layers are fixed to one another by any means and in particular by a layer of glue.

The textile material can be woven or non-woven and made of polyester or polypropylene fibers or of any other material used in the medical field and in particular for the production of survival blankets.

In the context of the invention this textile material is preferably non-woven.

The weight per unit area of the material is preferably between 15 and 500 g/m² and more preferably between 200 and 300 g/m².

The invention is not limited to this embodiment however and the sheet 1 could include a layer of textile material on each side of the heating element 10.

FIG. 2 also shows that the sheet 1 essentially includes four parts: a central and main part 12 of rectangular general shape with two longer sides 120 and 121 and two shorter sides 122 and 123, two lateral tabs 13a and 13b, and an elongate connecting zone 14.

Each of the lateral tabs 13a and 13b projects from a longer side 120, 121 of the central part 12 and is preferably linked to the central part 12 by a tear line 130a, 130b.

In the example depicted these two tabs are centered on the longer sides 120, 121.

On their face opposite the layer 11 of textile material each of the tabs 13a, 13b includes a layer 15a, 15b of adhesive protected by a film 16 that is peeled off when the sheet is placed in and fixed into a transport device. This is preferably an adhesive that enables the sheet to be torn at the level of the lines 130a, 130b without leaving residues after removing the lateral tabs. By way of example there may be cited a solvent-based acrylic adhesive.

In the example depicted in FIGS. 1 and 2 these two tabs have a substantially rectangular shape.

The connecting zone 14 projects relative to a shorter side 123 of the central part 12 and is continuous with the latter. This zone 14 has an elongate shape.

As FIG. 1 shows the heating element 10 and the covering layer 11 are the same shape. Each part of the protective sheet 1 that has just been described therefore includes within its thickness a part of the heating element 10 covered by a corresponding part of the covering layer 11.

The heating element 10 is in the form of a thin layer. In the example depicted in FIG. 1 it is a heating film including a metal conductive track 100 placed flat on an insulating film 101. It has a serpentine general shape on the central part 12 and is extended by two straight portions 102, 103 in the connecting zone 14. The track 100 can also include lateral portions like the lateral portion 106 in each of the tabs 13a, 13b.

Each of the portions 102, 103 of the connecting zone 14 has one end 104, 105 situated at the free end 140 of the connecting zone 14. These ends 104, 105 are therefore the two ends of the conductive track 100.

Near its free end 140 the connecting zone 14 includes positioning means 141 for electrical connection means which are described in more detail with reference to FIGS. 3 to 5. Here said positioning means consist in two perforations 141a, 141b produced through the thickness of the sheet 1 and therefore through the heating film 10 and the layer 11 of textile material. These perforations are situated between and parallel to the two aligned portions 102, 103. They are of no utility with the connector depicted in FIGS. 8 and 9.

The film 101 can for example be made of polyester and thus render the heating sheet impermeable.

The thickness of such a film is classically of the order of a few tens of microns.

This heating element is preferably flexible. This concept of flexibility is to be understood as the capability to adapt to three-dimensional surfaces without deterioration or malfunction.

The thickness of the protective heating sheet is generally between 0.2 mm and 3 mm and it is sufficiently flexible to fit the shape of a human body and even to be crumpled without losing its heating characteristics.

The conductive track 100 is made of a metal the resistance of which in the field of use of the heating sheet varies continuously and with no asymptotic behavior as a function of temperature and preferably in a linear or substantially linear manner.

This material is therefore different from the PTC materials used in the prior art, the resistance of which increases radically as a function of temperature beyond a particular temperature threshold fixed during the manufacture of the PTC material. This variation can be qualified as asymptotic because these materials are designed no longer to conduct electricity beyond this temperature threshold. They therefore have a switch function, unlike the heating element according to the invention.

The metal of the conductive track can for example be aluminum, copper, inconel or constantan.

Thanks to this feature it is possible to regulate the power supply to the heating element without needing technical means such as sensors in the heating element itself.

To this end the two straight parts 102, 103 of the conductive track are electrically connected to a control or regulation module 6 depicted in FIG. 6 by means of the electrical connector depicted in FIGS. 3 to 5 or FIGS. 8 and 9, this module being itself connected to an electrical power supply.

This module is designed to determine by itself the temperature of the heating sheet 1 at all times and to regulate the supply of electrical energy as a function of the comparison between the measured temperature and the required setpoint temperature.

This setpoint temperature is determined by the user as a function of the application and the environment and the module includes means enabling the user to program it.

Refer now to FIGS. 3 to 5 and in particular FIG. 3 which depicts the various components of the connector 3.

Thus the connector 3 includes a clip 4 and an electrical cable 5.

The clip 4 comprises two elements 40, 41 connected together by a hinge 42. It includes a pin 12a and means 12b for urging the clip into a closed position both retained in position by a nut 12c.

Each component 40, 41 includes a plane first part 400, 410 extended by a second part 401, 411 inclined relative to the first part. The first parts are intended to form together a jaw when the clip is closed, the second parts each forming a handle.

FIGS. 3 and 4 show that the first part 400 of the component 40 includes two blind openings 402a, 402b while the first part 410 of the component 41 includes two studs 412a, 412b on its internal face that face the openings 402a, 402b. FIG. 4 shows that these studs are placed in such a way as to be able to enter the blind openings when the clip 4 is closed.

The cable 5 includes two electrical wires 50, 51. At one end of the cable these wires are inserted in two parts 52, 53 respectively forming a positive terminal and a negative terminal. These parts are fixed in one of the first portions, here the first portion 41. Their positions are symmetrical relative to those of the blind openings 402a, 402b so that their distance relative to those openings corresponds to the distance between the portions 102, 103 of the conductive track and the perforations 141a, 141b of the zone 14.

At its other end the cable 5 is connected to a control module (not depicted).

In use, the open connector (depicted in chain-dotted line in FIG. 5) is disposed in the connecting zone 14 so that the zone 14 rests on the component 40 and the openings 402a,402b coincide with the two perforations 141a, 141b in the zone 14.

The clip 4 is then closed by the operator to assume the configuration depicted in FIG. 4 or the configuration depicted in solid line in FIG. 5. In this configuration the tabs 412a, 412b of the first part 410 of the component 41 can pass through the perforations 141a, 141b in the zone 14 and enter the openings 402a, 402b.

In this closed configuration each of the two terminals 52, 53 comes into electrical contact with one of the straight portions 102, 103 at the level of the ends 104 and 105 of the conductive track. As FIG. 6 shows the heating sheet 1 is then electrically connected to the control module.

FIGS. 8 and 9 depict a variant embodiment of the connector between the heating sheet 1 and the electrical cable 5 connected to the module 6.

This connector 9 includes a female part 90 connected to the module 6 (not depicted). It forms a housing providing a slot 900 and a cavity 901 in which two electrical wires 50, 51 of the cable 5 are fixed. The end of each wire forms an electrical contact that extends in the slot 900. Only the end 500 is depicted in FIG. 9.

The connector 9 also includes a male part 91 corresponding to the end portion of the connecting zone 14. This part 91 consists of a portion of the zone 14 near its free end 140 reinforced by a thin layer of polymer. By way of example a polyester layer 250 microns thick is sufficient to stiffen this part 91.

This latter part includes the ends 104, 105 of the conductive track 100.

Between these two ends the part 91 includes an elongate opening 910 that opens onto the free end 140 and which grips a stud 902 situated in the part 90 when the part 91 is inserted in the slot 900 in the direction of the arrow F. FIG. 8 shows that the part 91 also includes a notch 911 on one of its edges that serves as a polarizer and assists the user to insert this part 91 the right way round.

In fact the connection between the heating sheet 1 and the module 6 is made by inserting the male part 91 in the slot 900 of the module so that the electrical contacts bear on the ends 104, 105 to make the electrical connection. The polarizer prevents the user from inserting the part 91 upside down, in which position the electrical connection would not be made.

The heating sheet 1 and the module 6 are easily disconnected by pulling the female part out of the slot in the opposite direction to the arrow F.

Of course the invention is not limited to the embodiments described. Thus the connecting zone of the heating sheet could be longer so as to position the connector outside the device. Also a connector need not be used and the heating sheet could be equipped with electrical wires connected directly to the control module. This latter solution would be more costly however, since the electrical wires would also be for single use.

FIG. 6 depicts the heating sheet 1 electrically connected to the control module 6, in particular by means of the connector 3 that has just been described with reference to FIGS. 3 to 5.

The module 6 is supplied with electrical energy by means of a battery 70 intended to be connected to an electrical power supply via a charger 71 of appropriate voltage, for example 110 V, 230 V or 12 V. The invention is not limited to this example and the heating sheet could be connected directly to an electrical power supply via the control module.

Battery power is nevertheless more suitable if the sheet 1 is used with a transport device.

Operation is described in more detail next with the sheet 1 connected to the module, itself supplied with electrical energy by the battery 70.

Firstly, in an initialization step the module measures the current between the terminals of the heating element 10 of the heating sheet 1 and deduces therefrom the initial resistance (R0) of the sheet and current (I0) in the sheet.

The module also measures the ambient temperature (T0), which corresponds to the initial temperature of the sheet, the latter also being at ambient temperature.

Once this initialization step has been carried out the heating element 10 is supplied with electrical energy by the module and after a particular time interval (t) the module again measures the current (It) at the terminals of the heating element to deduce therefrom the value of its resistance (Rt) and thus its temperature (Tt) at time t.

The module then compares the temperature t (Tt) and the setpoint temperature (Tc) set beforehand by the operator.

In this respect the control module offers the operator the possibility of setting the setpoint temperature to a value chosen in a particular range, generally between 30° C. and 41° C. and preferably between 30° C. and 37° C.

If this comparison reveals that the temperature Tt at time t is lower than the setpoint temperature Tc the module continues to supply the heating element with electrical energy.

If not the control module interrupts the supply of electrical energy so that the temperature of the heating sheet can fall toward the setpoint temperature.

To maintain a constant temperature the temperature is checked at regular time intervals, generally every 0.01 to 1 second, the measuring time being a few milliseconds.

This control is very precise because it is based on a voltage measurement that gives the exact temperature of the heating sheet. This is not the case for the control method used by the known means employing heating materials that exhibit a sudden change of resistance at a given temperature. In fact, the characteristics of those materials can evolve over time and no longer provide the required accuracy, especially in the medical field.

Over and above this the value of the setpoint temperature can be changed, which is not the case with these materials that are designed for self-regulation of temperature at a setpoint temperature that cannot be varied.

Refer now to FIG. 7, which depicts an example of a device for transporting newborns.

This device 8 includes a first part 80 and a second part 81. This second part (not depicted) is removable and can be fixed to the first part to delimit an enclosure. The first part 80 forms a housing for a newborn and the second part 81 is a preferably transparent cover.

Padding or an inflatable mattress 800 is disposed inside this housing 80 to ensure that the newborn is comfortable.

FIG. 7 shows a heating sheet 1 according to the invention disposed on this padding 800. To retain the sheet on this padding the film 16 is removed from each of the tabs 13a, 13b to free the layers 15a, 15b of adhesive and apply them to the padding.

FIG. 7 also depicts a connector 3 the clip 4 of which is positioned at the end of the connecting zone 14 of the heating sheet 1, and the cable 5 is therefore connected to a control module 6. The cable passes through one of two openings 801, 802 in the wall of the housing 80. This figure also depicts the battery 7 connected to the module 6 to supply it and likewise the sheet 1 with electrical energy.

This FIG. 7 shows that in use the connecting zone 14 enables the connector 3 to be kept away from the newborn and therefore prevents all risk of injury by contact with this component. The situation is exactly the same with the connector 9 depicted in FIGS. 8 and 9.

When the sheet 1 is removed from the padding it breaks along the tear lines 130a, 130b. The central part 12 and the connecting zone 14 of the sheet are detached from the tabs 13a, 13b, which are removed afterwards. This prevents any re-use of the sheet, which is then necessarily for single use.

When the heating sheet according to the invention is supplied with electrical energy heat is generated as close as possible to the transported patient with no losses. This is not the case if the heat is generated outside the transport device and then transferred to the vicinity of the patient.

Unlike known devices the transport device depicted in FIG. 7 does not necessitate forced ventilation with warm air to heat the enclosure and therefore to maintain the temperature of the newborn. The heating sheet according to the invention therefore also avoids any stirring up of dust caused by movement of air.

The invention is not limited to the application that has just been described and the heating sheet according to the invention could be used in static devices, such as incubators for newborns.

1. A protective heating sheet intended in use to be removably fixed into a device for maintaining the temperature of a human being and in particular a newborn, including a thin layer heating element (10) with flat electrically-conductive means and at least one layer (101) made of an electrically-insulative material intended to be connected to an electrical power supply, wherein said electrically-conductive means are made of a conductive material the resistance of which varies continuously as a function of temperature, at least one face of said heating element being at least partly covered by a textile material covering layer (11).

2. The protective heating sheet as claimed in claim 1 including at least one fixing tab (13a, 13b) in said temperature maintaining device linked to a central and main part (12) of the sheet and projecting from said main part, said tab having a face provided with a layer (15a, 15b) of adhesive.

3. The protective heating sheet as claimed in claim 1 for single use.

4. The protective heating sheet as claimed in claim 2 wherein said at least one fixing tab (13a, 13b) is linked to said main part (12) by a tear line (130a, 130b).

5. The protective heating sheet as claimed in claim 2 including a connecting zone (14) to be connected to said electrical power supply, linked to said main part (12) and projecting relative to said main part, a conductive track (100) extending over this connecting zone and ending at two ends (104, 105).

6. The protective heating sheet as claimed in claim 5 wherein the connecting zone (14) includes means (141) for positioning an electrical connector (3).

7. The protective heating sheet as claimed in claim 1 wherein the heating element is a heating film, the electrically-conductive means consisting in a metal conductive track (100) positioned at least on said central part (12) of the heating sheet.

8. The protective heating sheet as claimed in claim 1 which is sterile.

9. A kit comprising at least one protective heating sheet (1) as claimed in claim 1 and an electrical power supply control module (6) intended to be connected between the heating sheet and an electrical power supply, said module including means for determining the resistance of the conductive means of the heating element, deducing therefrom the temperature (Tt) of the woven material at a time t, comparing it to a setpoint value (Tc), and controlling the electrical power supply as a function of said comparison.

10. The kit as claimed in claim 9 including an electrical connector (3) between said heating sheet (1) and said module (6), said connector being removable.

11. A device for maintaining the temperature of human beings and in particular of newborns, including two parts (80, 81) delimiting an enclosure, one forming a housing for a human being and including a protective heating sheet as claimed in claim 1 disposed in said housing.

12. A device for maintaining the temperature of human beings and in particular newborns, in particular a transport device, comprising two parts (80, 81) delimiting an enclosure, one forming a housing for a human being and including a kit as claimed in claim 9, said protective heating sheet being disposed in said housing and said module being outside said enclosure.

13. The device as claimed in claim 11 including padding, said protective sheet being removably fixed to said padding.

14. The use of a protective heating sheet as claimed in claim 1 in a device, notably a transport device, for maintaining the temperature of human beings and in particular of newborns.
